(19) United States
(12) Patent Application Publication
(10) Pub. No.: US 2015/0289838 A1
(43) Pub. Date: Oct. 15, 2015

(54) SYSTEMS AND METHODS FOR REAL-TIME ASSESSMENT OF THE PRESENCE AND QUANTITY OF CAROTID BLOOD FLOW DURING CARDIAC ARREST

(71) Applicant: UNIVERSITY OF WASHINGTON THROUGH ITS CENTER FOR COMMERCIALIZATION, Seattle, WA (US)

(72) Inventors: Graham Nichol, Mercer Island, WA (US); Adeyinka Adedipe, Seattle, WA (US)

(21) Appl. No.: 14/438,131

(22) PCT Filed: Oct. 25, 2013
(86) PCT No.: PCT/US2013/066976

§ 371 Date: Apr. 23, 2015

Related U.S. Application Data
(60) Provisional application No. 61/718,845, filed on Oct. 26, 2012.

(51) Int. Cl. A61B 8/06; A61B 8/00; A61B 8/04

(52) U.S. Cl. CPC A61B 8/065; A61B 8/04; A61B 8/4263; A61B 8/46

(57) ABSTRACT

The present technology relates generally to blood flow measuring systems and associated devices and methods. In some embodiments, a system for measuring blood flow during a cardiac arrest configured in accordance with the technology comprises an interface element configured to be removably attached to a patient's skin at a target site proximate to a carotid artery of the patient, a hand-held ultrasound transducer configured to be positioned in contact with the interface element at the target site, and a controller operably coupled to the ultrasound transducer and configured to determine a blood flow velocity of the patient based on received ultrasound data. In some embodiments, the adhesive member can include one or more reference indicia corresponding to anatomical landmarks associated with a desired position of the ultrasound transducer relative to the carotid artery.

### CROSS-REFERENCE TO RELATED APPLICATION(S)

This application claims the benefit of U.S. Provisional Patent Application No. 61/718,845, filed Oct. 26, 2012, which is incorporated herein by reference in its entirety.

### TECHNICAL FIELD

The present technology is generally related to ultrasound devices and associated systems and methods. In particular, several embodiments are directed to ultrasound devices and associated methods for measuring carotid blood flow.

### BACKGROUND

Conventional techniques for blood flow monitoring during time-sensitive medical emergencies, such as cardiac arrest, suffer from several drawbacks. For example, to determine the need for cardiopulmonary resuscitation (“CPR”) in the setting of a cardiac arrest, lay persons and emergency personnel typically attempt to palpate a major artery (e.g., the carotid or femoral artery) for the presence or absence of a pulse. In many cases, however, such approaches are undesirable and/or inaccurate because the patient can be violently and/or abruptly moving. As a consequence, potentially life-saving CPR may be withheld from those individuals who could have benefitted from CPR but were not recognized as being in cardiac arrest. In addition, many existing non-invasive blood flow monitoring devices require application to the chest wall, which is likely to interfere with attempted CPR.

To address the challenges associated with non-invasive blood flow monitoring methods, several invasive monitoring devices and approaches exist, such as pulmonary artery catheters and lithium dilution. Such techniques, however, are generally not suitable for application during emergency cardiac arrest because (1) the insertion of an invasive device necessarily interrupts ongoing CPR; (2) invasive blood flow monitoring comes with an increased risk of vascular injury, infection or other adverse events; and (3) invasive monitors that require a wire- or catheter-based technology may be sensitive to movement so that even subtle changes in position (such as those occurring during chest compressions) can result in inaccurate readings because the device is inadvertently moved from the proper position to measure blood flow.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. Furthermore, components can be shown as transparent in certain views for clarity of illustration only and not to indicate that the illustrated component is necessarily transparent.

FIG. 1 is a schematic representation of a blood flow measuring system configured in accordance with the present technology.

FIGS. 2A-2C illustrate a method for measuring blood flow using the ultrasound transducer of FIG. 1 in accordance with embodiments of the present technology.

FIGS. 3A and 3B are perspective views of interface elements configured in accordance with the present technology.

FIG. 4 is a side perspective view of an interface element having indicia configured in accordance with the present technology.

FIG. 5 is a display diagram of an ultrasound image obtained using an embodiment of the system of FIG. 1.

### DETAILED DESCRIPTION

The present technology is generally directed to devices, systems, and methods for non-invasively measuring blood flow. In one embodiment, for example, a blood flow measuring system includes a patient assembly configured to be rapidly positioned and stabilized at an external location proximate to a major artery of a human patient while the patient is experiencing cardiac arrest. The system can measure blood flow, for example, to identify the onset of cardiac arrest before it occurs, verify the presence or absence of cardiac arrest, guide CPR efforts during cardiac arrest, to achieve specific hemodynamic or physiologic targets, and/or assess cardiac arrest prognosis during or immediately after cardiac arrest.

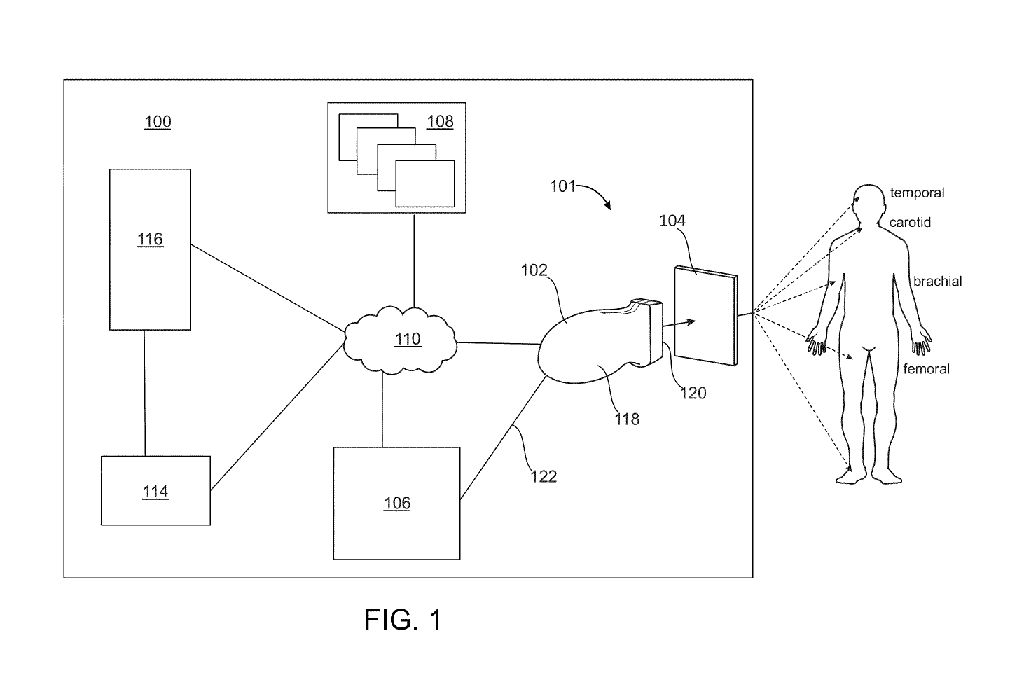

Specific details of several embodiments of the present technology are described herein with reference to FIGS. 1-5. Although many of the embodiments are described below with respect to devices, systems, and methods for measuring blood flow via the carotid artery using ultrasound, other physiologic parameter measuring applications are within the scope of the present technology such as blood pressure monitoring, blood flow monitoring at other major arteries (e.g., femoral, brachial, temporal, etc.), etc. Additionally, other embodiments of the present technology can have different configurations, components, or procedures than those described herein. For example, other embodiments can include additional elements and features beyond those described herein, or other embodiments may not include several of the elements and features shown and described herein.

For ease of reference, throughout this disclosure identical reference numbers are used to identify similar or analogous components or features, but the use of the same reference number does not imply that the parts should be construed to be identical. Indeed, in many examples described herein, the identically-numbered parts are distinct in structure and/or function.

Generally, unless the context indicates otherwise, the terms “distal” and “proximal” define a position or direction with respect to the treating clinician or clinician's control device (e.g., an ultrasound device). “Distal” or “distally” can refer to a position distant from or in a direction away from the clinician or clinician's control device. “Proximal” and “proximally” can refer to a position near or in a direction toward the clinician or clinician's control device.

### Selected Embodiments of Blood Flow Monitoring Devices, Systems, and Methods

FIG. 1 is a schematic representation of a non-invasive blood flow monitoring system 100 (“system 100”) configured in accordance with the present technology. The system 100 can include a patient assembly 101 having an ultrasound transducer 102 and an interface element 104. The ultrasound transducer 102 is configured to be positioned on a human patient at an external location proximate to a major artery (e.g., a carotid artery), and the interface element 104 is configured to be positioned between at least a portion of the ultrasound transducer 102 and the patient's skin. In some embodiments, the system 100 can include more than one ultrasound transducer 102 and/or interface element 104. During a time-sensitive medical emergency, such as cardiac arrest, the ultrasound transducer 102 can be rapidly positioned and stabilized at an external location proximate the targeted artery via the interface element 104.

As described in greater detail below, the system 100 can also include a controller 106 configured to measure, analyze, and/or indicate the patient's blood flow velocity and/or blood pressure in real time to guide CPR efforts during cardiac arrest and/or assess the patient's prognosis. In some embodiments, measurements obtained by the patient assembly 101 can be mapped to electrocardiographic (“EKG”) recordings so that the presence of a perfusing cardiac rhythm can be differentiated from pulseless electrical activity. In further embodiments (and as mentioned below) the controller 106 can determine the blood pressure via ultrasound measurements measured at two proximate locations in the targeted artery. Additionally or alternatively, in some embodiments, the vessel diameter can be measured, and the vessel diameter measurements and blood velocity measurements can be used to derive an estimate of blood pressure in the vessel.

As shown in FIG. 1, the ultrasound transducer 102 can have a body portion 118 shaped to be easily and comfortably held and manipulated by a health care provider (“HCP”) (not shown). At least a portion of a distal surface 120 of the transducer 102 is configured to be positioned in contact with the interface element 104. In some embodiments the ultrasound transducer 102 comes with the interface element 104 pre-associated with the distal surface 120 of the transducer 102. In other embodiments, the transducer 102 may be rapidly connected to the interface element 104 at the time of use.

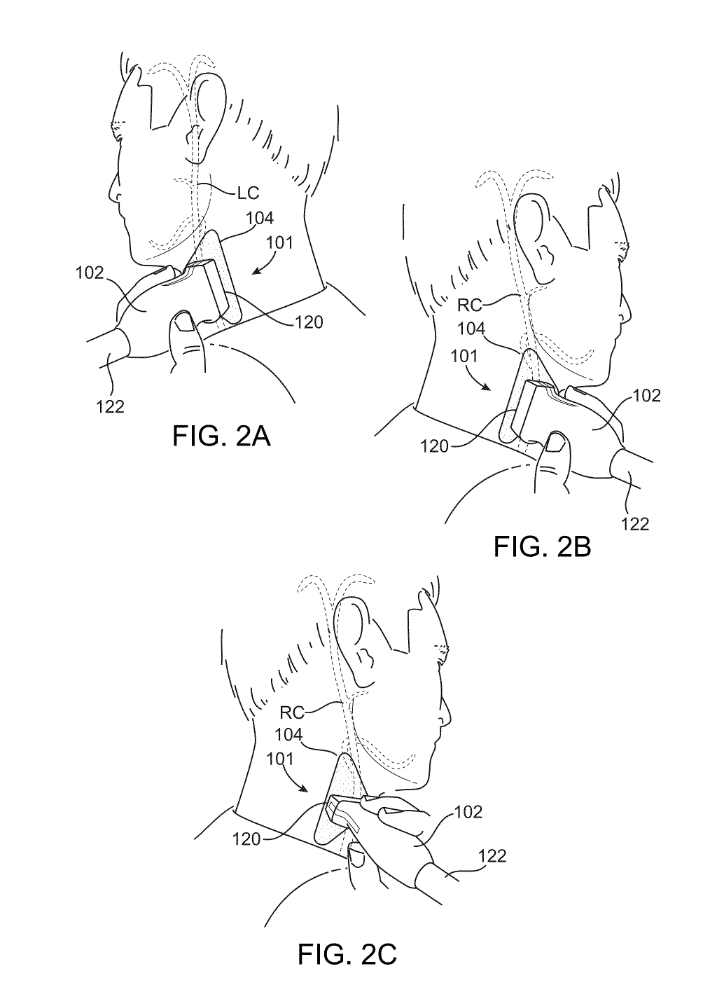

FIGS. 2A-2C illustrate various methods for measuring blood flow using the patient assembly 101. Although FIGS. 2A-2C show the transducer 102 positioned over the carotid arteries, as noted above, in other embodiments the transducer 102 can be positioned over other suitable major arteries of the patient (e.g., femoral, temporal, brachial, etc.). In embodiments where the interface element 104 is pre-associated with the distal surface 120 of the transducer 102, the transducer 102 can be immediately applied to the skin proximate the targeted artery. In embodiments where the interface element 104 is separate from the transducer 102, the interface element 104 can first be applied to the skin proximate the targeted artery and then the transducer 102 may be brought into contact with the interface element 104.

Referring first to FIG. 2A, the patient assembly 101 can be positioned in a vertical or generally vertical configuration at an external location proximate a left carotid artery LC of the patient P. As described in greater detail below, the interface element 103 can be designed to hold the transducer 102 in a generally stationary position relative to the patient's skin throughout a desired measurement period. As a result, the patient assembly 101 of the present technology is expected to provide reliable, accurate blood flow measurements despite sharp or unexpected patient movement (as is often the case during cardiac arrest and/or CPR).

As shown in FIG. 2B, the patient assembly 101 may also be positioned in a vertical configuration at an external location proximate a right carotid artery RC of the patient P. Although only a single transducer 102 is shown in the illustrated embodiment, in some embodiments (not shown), a first transducer can be positioned at the left carotid artery LC while a second transducer is simultaneously positioned at the right carotid artery RC. Referring next to FIG. 2C, in some embodiments the patient assembly 101 can be positioned in a horizontal or generally horizontal configuration at an external location proximate the left/right carotid arteries LC/RC of the patient P.

Referring to FIGS. 1-2C together, the ultrasound transducer 102 can be configured to transmit and receive ultrasound waves to and from the targeted artery (e.g., the left carotid artery LC and/or right carotid artery RC) from the external location. The transducer 102 can emit and receive ultrasound waves to and from the targeted artery intermittently or continuously. In some embodiments, the transducer 102 initially emits ultrasound waves at a first frequency f₀. When waves at the first frequency f₀ strike a moving object(s) within the targeted artery (such as red blood cells), the reflected ultrasound waves return to the transducer 102 at a second frequency f₁ that is different than the first frequency f₀. The controller 106 (FIG. 1) detects this change in frequency (f₀-f₁) and determines the blood flow velocity utilizing the Doppler equation. Control of the timing and frequency of the emitted ultrasound waves can be automated (e.g., via the controller) or can be manual (e.g., by the HCP).

Referring back to FIG. 1, the controller 106 can be a separate device coupled to the ultrasound transducer 102 via a connector 122 (e.g., a cable) or wirelessly (e.g., Bluetooth, RF, RFID, electromagnetic waves, infrared, etc.). The controller 106 can be configured to transmit and receive signals to and from the transducer 102. The controller 106 can comprise, for example, a personal computer(s), server computer(s), handheld or laptop device(s), multiprocessor system(s), microprocessor-based system(s), programmable consumer electronic(s), digital camera(s), network PC(s), minicomputer(s), mainframe computer(s), tablets, and/or any suitable computing environment. Additionally or alternatively, the controller 106 can be part of the ultrasound transducer 102.

The controller 106 can include memory (not shown), storage devices (e.g., disk drives), one or more output devices (e.g., a display), one or more input devices (e.g., a keyboard, a touchscreen, etc.) and processing circuitry (not shown). The memory and storage devices are computer-readable storage media that may be encoded with non-transitory, computer-executable instructions. In addition, the instructions, data structures, and message structures may be stored or transmitted via a data transmission medium, such as a signal on a communications link and may be encrypted. Various communications links may be used, such as the Internet, a local area network, a wide area network, a point-to-point dial-up connection, a cell phone network, Bluetooth, RFID, and other suitable communication channels. Aspects of the system can also be practiced in distributed computing environments where tasks or modules are performed by remote processing devices, which are linked through a communications network, such as a Local Area Network (LAN), Wide Area Network (WAN), Storage Area Network (SAN), Fibre Channel, or the Internet. In a distributed computing environment, program modules may be located in both local and remote memory storage devices.

In some embodiments, the controller 106 can provide real-time feedback to the HCP via an indicator (not shown). Such indicators can include one or more display(s), user interface(s), LEDs, speaker(s), and/or other similarly communicative devices. For example, the controller 106 may include a graphical user interface that can receive HCP input and/or provide blood flow information to the HCP. The feedback can guide an HCP in administering CPR and/or determine the effectiveness of any ongoing CPR or medical efforts.

Additionally, the controller 106 can be in communication with a wired or wireless network 110 so that blood flow measurements can be remotely available in real-time to HCPs. For example, the network 110 can actively communicate information from the controller 106 to other devices 108 on the network 110, such as personal computers in the doctor's lounge, nurse's station, etc. In particular embodiments, the controller 106 and/or network 110 can communicate with a server 116 (e.g., via the Internet) so that ultrasound data can be available outside of the network 110. For example, ultrasound data can be available on a home computer, a smart phone, a tablet, a personal computer within another network (e.g., at a different medical care center), and/or other remote devices. In these and other embodiments, blood flow measurements can be stored in a central database and accessed later for analysis. Further, it will be appreciated that other configurations and communication channels can be used to provide remote access and/or monitoring.

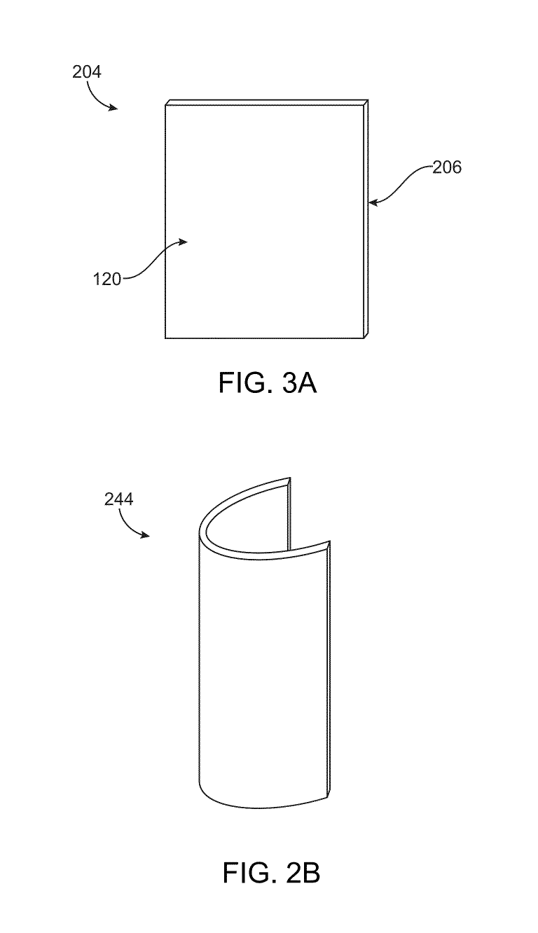

FIGS. 3A and 3B are perspective views of interface elements configured in accordance with various embodiments of the present technology. FIG. 3A, for example, illustrates one embodiment of an interface element 204 defined by a generally square or rectangular flexible pad. The interface element 204 can have distal surface 206 configured to be positioned in direct contact with or proximate to a patient's skin, and a proximal surface 208 configured to receive the distal surface 120 of the transducer 102 (FIG. 1). The proximal 208 and/or distal surface 206 can include a medical-grade adhesive adapted to hold the interface element 204 in place on the patient's skin such that violent, abrupt, repeated patient motion (present during cardiac arrest and/or CPR) will not disturb the position of the distal surface 120 of the transducer 102 relative to the skin. FIG. 3B illustrates another embodiment of an interface element 244 configured in accordance with the present technology. As shown in FIG. 3B, the interface element 244 can have a curved profile so as to better conform to the patient's anatomy proximate the targeted artery. In a particular embodiment (not shown), a proximal surface 248 of the interface element 244 can include one or more stabilizing features (not shown) to further stabilize placement of the transducer 102 with respect to the interface element 244 and patient. In other embodiments, the interface elements 204 and 244 may have other suitable shapes (e.g., circular, triangular (FIG. polygonal, etc.), sizes, and/or configurations based, at least in part, on the targeted patient anatomy onto which the interface elements will be delivered.

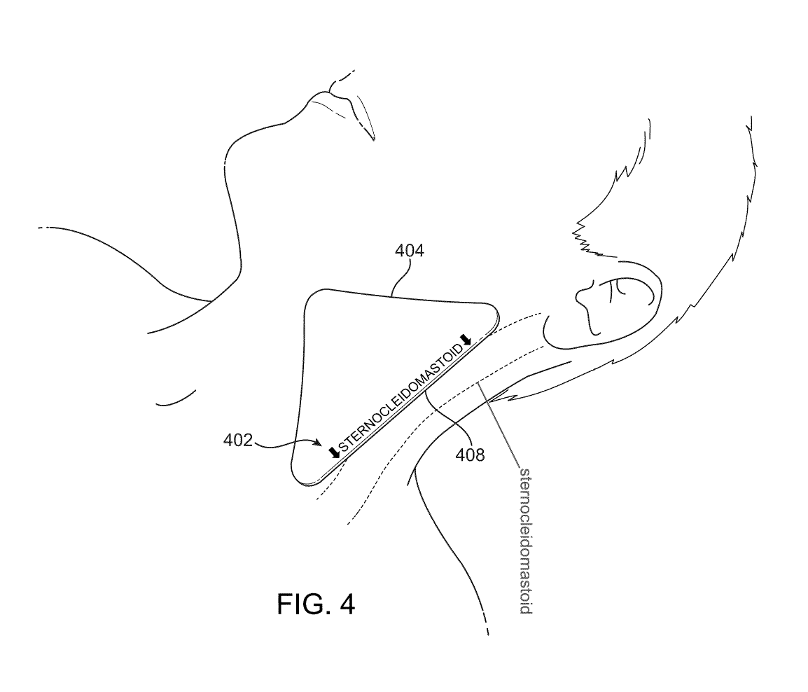

FIG. 4 is a perspective view of another embodiment of an interface element 404 configured in accordance with the present technology. As shown in FIG. 4, the interface element 404 can include one or more reference indicia 402 that correspond to anatomical landmarks proximate to the targeted artery. The anatomical landmarks can be identifiable by visual inspection from an external location and/or by feeling the anatomy at or near the targeted artery. The indicia 402 can include markings, bumps, grooves, cuts, and/or other features suitable to guide an HCP in positioning the transducer 102 on the interface element 404, and/or positioning the interface element 404 on the patient's skin. In some embodiments, the indicia 402 can correspond to anatomical landmarks associated with a desired position of the ultrasound transducer relative to the targeted artery. For example, in the illustrated embodiment, the interface element 404 is intended for placement proximate the carotid artery. As a result, the indicia 402 can include text (e.g., “STERNOCLEIDOMASTOID”) or markings (e.g., ↓) that guide positioning of the interface element 404 so that a first edge 408 of the element 404 is positioned along a sternocleidomastoid muscle of the patient. In other embodiments, the indicia 402 can correspond to anatomical landmarks associated with other arteries, such as the femoral and temporal arteries. For example, for interface elements 404 configured to be placed over a femoral artery, the indicia can correspond to a sartorius muscle of the patient.

In operation, when the ultrasound transducer 102 (FIG. 1) is positioned on the body according to the indicia 402, the ultrasound transducer 102 can rapidly localize arterial blood flow. Additional embodiments of the present technology may include indicia to guide positioning of the transducer and/or interface element over the femoral artery, radial artery, brachial artery, and/or other suitable arteries. In these and other embodiments, the interface elements can have any size or shape suitable for adherence to a particular portion of the body and sized appropriately to accommodate the necessary indicia for the HCP.

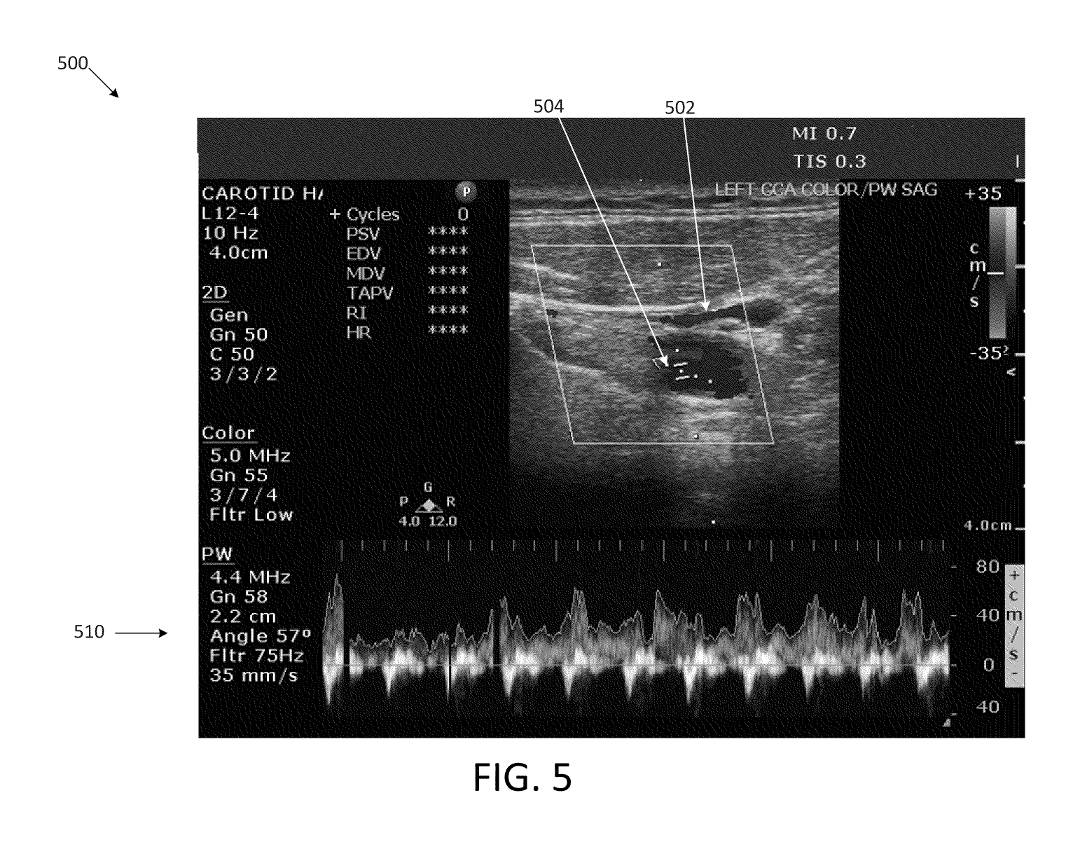

FIG. 5 is a display diagram 500 of an ultrasound image obtained during cardiac arrest using an embodiment of the system of FIG. 1. As shown in FIG. 5, the display diagram 500 can include an ultrasound image showing a transverse view of the internal jugular vein 504 and the carotid artery 502. In some embodiments, the display diagram can additionally or alternatively include a longitudinal view and/or other suitable views and/or other anatomical structures of interest. The system 100 can include color Doppler analysis that is configured to display a first color indicating blood flow toward the ultrasound transducer (FIGS. 1-3C) and a second color flow (different than the first color) indicating blood flow away from the ultrasound transducer. This and other analytic tools are used to aid HCPs in confirming the presence, absence, and/or velocity of blood flow in the targeted artery during chest compressions (e.g. during cardiac arrest). In some embodiments, representative blood velocities 510 are displayed on the screen. Additionally, blood pressure (not shown) can also be determined and displayed.

### Examples

1. A system for monitoring blood flow of a human patient during cardiac arrest, the system comprising:

a hand-held ultrasound transducer;

an interface element configured to be removably attached to the patient's skin at a target site proximate to a carotid artery of the patient, wherein the adhesive member includes one or more reference indicia corresponding to anatomical landmarks associated with a desired position of the ultrasound transducer relative to the carotid artery,

wherein the ultrasound transducer is configured to be positioned in contact with the interface element at the target site; and

a controller operably coupled to the ultrasound transducer and configured to—
receive ultrasound data from the ultrasound transducer;
determine a blood flow velocity of the patient based on the ultrasound data.

2. The system of example 1 wherein the reference indicia correspond to a projected position and orientation of the carotid artery within the patient.

3. The system of example 1 wherein the system further comprises an indicator operably coupled to the controller, and wherein the indicator is configured to provide audio, visual, and/or haptic feedback regarding the blood flow velocity to a user.

4. The system of example 1 wherein the ultrasound transducer is a first ultrasound transducer and the interface element is a first interface element, and wherein the system further comprises a second ultrasound transducer operably coupled to the controller and a second interface element, and further wherein, during operation, the first interface element and first ultrasound transducer are configured for placement proximate a left carotid artery of the patient, and the second interface element and second ultrasound transducer are configured for placement proximate a right carotid artery of the patient.

5. A method for measuring blood flow within a carotid artery of a human patient during a cardiac arrest event, the method comprising:

positioning an adhesive member on skin of the patient at a target location proximate the carotid artery;

positioning an ultrasound transducer in contact with the adhesive member such that at least a portion of the adhesive member is between the ultrasound transducer and the skin of the patient; and

determining a blood flow measurement within the carotid artery of the patient via the ultrasound transducer.

6. The method of example 5 wherein positioning the adhesive member on skin of the patient comprises positioning the adhesive member proximate to a left carotid artery of the patient.

7. The method of example 5 wherein positioning the adhesive member on skin of the patient comprises positioning the adhesive member proximate to a right carotid artery.

8. The method of example 5 wherein the adhesive member is a first adhesive member positioned at a first target location proximate a left carotid artery of the patient, the ultrasound transducer is a first ultrasound transducer, and determining a blood flow measurement comprises determining the blood flow within the left carotid artery via the first ultrasound transducer, and wherein the method further comprises:

positioning a second adhesive member on skin of the patient at a second target location proximate a right carotid artery of the patient;

positioning a second ultrasound transducer in contact with the second adhesive member; and

determining a blood flow measurement within the right carotid artery of the patient via the second ultrasound transducer.

9. The method of example 8 wherein determining the blood flow measurement within the left carotid artery via the first ultrasound transducer and determining the blood flow measurement within the right carotid artery via the second ultrasound transducer occur simultaneously.

10. The method of example 8 wherein determining the blood flow measurement within the left carotid artery via the first ultrasound transducer and determining the blood flow measurement within the right carotid artery via the second ultrasound transducer occur in sequence.

11. The method of example 5, further comprising transmitting the determined blood flow measurements to a remote device.

12. The method of example 5, further comprising providing audio, visual, and/or haptic feedback to a health care provider regarding the blood flow measurement.

13. The method of example 5, further comprising determining a blood pressure of the patient based, at least in part, on the determined blood flow measurement.

14. The method of example 5, further comprising:

determining a diameter of the artery; and

determining a blood pressure of the patient based, at least in part, on the determined blood flow measurement and the determined artery diameter.

15. An interface element for use with an ultrasound transducer, the interface element comprising:

a body portion including—
a first surface configured to be positioned on skin of a human patient proximate to a major artery of the patient; and
a second surface configured to engage the ultrasound transducer; and

a first adhesive material at the distal surface;

a second adhesive material at the proximal surface;

one or more reference indicia at the proximal surface, wherein the indicia correspond to (a) an anatomical landmark associated with a projected position and/or orientation of the major artery of the patient, and (b) a desired orientation for placement of the ultrasound transducer relative to the major artery.

16. The interface element of example 15 wherein the reference indicia comprise at least one of a marking, bump, groove and/or cut in the interface element.

17. The interface element of example 15 wherein the reference indicia correspond to the carotid artery.

18. The interface element of example 15 wherein the reference indicia correspond to the femoral artery.

19. The interface element of example 15 wherein the reference indicia correspond to the temporal artery.

20. The interface element of example 15 wherein the reference indicia correspond to the brachial artery.

### Conclusion

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/we claim:

1. A system for monitoring blood flow of a human patient during cardiac arrest, the system comprising:
a hand-held ultrasound transducer;
an interface element configured to be removably attached to the patient's skin at a target site proximate to a carotid artery of the patient, wherein the adhesive member includes one or more reference indicia corresponding to anatomical landmarks associated with a desired position of the ultrasound transducer relative to the carotid artery,
wherein the ultrasound transducer is configured to be positioned in contact with the interface element at the target site; and
a controller operably coupled to the ultrasound transducer and configured to—
receive ultrasound data from the ultrasound transducer;
determine a blood flow velocity of the patient based on the ultrasound data.

2. The system of claim 1 wherein the reference indicia correspond to a projected position and orientation of the carotid artery within the patient.

3. The system of claim 1 wherein the system further comprises an indicator operably coupled to the controller, and wherein the indicator is configured to provide audio, visual, and/or haptic feedback regarding the blood flow velocity to a user.

4. The system of claim 1 wherein the ultrasound transducer is a first ultrasound transducer and the interface element is a first interface element, and wherein the system further comprises a second ultrasound transducer operably coupled to the controller and a second interface element, and further wherein, during operation, the first interface element and first ultrasound transducer are configured for placement proximate a left carotid artery of the patient, and the second interface element and second ultrasound transducer are configured for placement proximate a right carotid artery of the patient.

5. A method for measuring blood flow within a carotid artery of a human patient during a cardiac arrest event, the method comprising:
positioning an adhesive member on skin of the patient at a target location proximate the carotid artery;
positioning an ultrasound transducer in contact with the adhesive member such that at least a portion of the adhesive member is between the ultrasound transducer and the skin of the patient; and
determining a blood flow measurement within the carotid artery of the patient via the ultrasound transducer.

6. The method of claim 5 wherein positioning the adhesive member on skin of the patient comprises positioning the adhesive member proximate to a left carotid artery of the patient.

7. The method of claim 5 wherein positioning the adhesive member on skin of the patient comprises positioning the adhesive member proximate to a right carotid artery.

8. The method of claim 5 wherein the adhesive member is a first adhesive member positioned at a first target location proximate a left carotid artery of the patient, the ultrasound transducer is a first ultrasound transducer, and determining a blood flow measurement comprises determining the blood flow within the left carotid artery via the first ultrasound transducer, and wherein the method further comprises:
positioning a second adhesive member on skin of the patient at a second target location proximate a right carotid artery of the patient;
positioning a second ultrasound transducer in contact with the second adhesive member; and
determining a blood flow measurement within the right carotid artery of the patient via the second ultrasound transducer.

9. The method of claim 8 wherein determining the blood flow measurement within the left carotid artery via the first ultrasound transducer and determining the blood flow measurement within the right carotid artery via the second ultrasound transducer occur simultaneously.

10. The method of claim 8 wherein determining the blood flow measurement within the left carotid artery via the first ultrasound transducer and determining the blood flow measurement within the right carotid artery via the second ultrasound transducer occur in sequence.

11. The method of claim 5, further comprising transmitting the determined blood flow measurements to a remote device.

12. The method of claim 5, further comprising providing audio, visual, and/or haptic feedback to a health care provider regarding the blood flow measurement.

13. The method of claim 5, further comprising determining blood pressure of the patient based, at least in part, on the determined blood flow measurement.

14. The method of example 5, further comprising:
determining a diameter of the artery; and
determining a blood pressure of the patient based, at least in part, on the determined blood flow measurement and the determined artery diameter.

15. An interface element for use with an ultrasound transducer, the interface element comprising:
a body portion including—
a first surface configured to be positioned on skin of a human patient proximate to a major artery of the patient; and
a second surface configured to engage the ultrasound transducer; and
a first adhesive material at the distal surface;
a second adhesive material at the proximal surface;
one or more reference indicia at the proximal surface, wherein the indicia correspond to (a) an anatomical landmark associated with a projected position and/or orientation of the major artery of the patient, and (b) a desired orientation for placement of the ultrasound transducer relative to the major artery.

16. The interface element of claim 15 wherein the reference indicia comprise at least one of a marking, bump, groove and/or cut in the interface element.

17. The interface element of claim 15 wherein the reference indicia correspond to the carotid artery.

18. The interface element of claim 15 wherein the reference indicia correspond to the femoral artery.

19. The interface element of claim 15 wherein the reference indicia correspond to the temporal artery.

20. The interface element of claim 15 wherein the reference indicia correspond to the brachial artery.
